(19) United States
(12) Patent Application Publication
(10) Pub. No.: US 2007/0071696 A1
(43) Pub. Date: Mar. 29, 2007

(54) Dual phase whitening dentifrice

(75) Inventors: Qin Wang, Monmouth Junction, NJ (US); Suman K. Chopra, Dayton, NJ (US); Lynette Zaidel, Cranford, NJ (US); Michael Prencipe, West Windsor, NJ (US)

Correspondence Address: COLGATE-PALMOLIVE COMPANY, 909 RIVER ROAD, PISCATAWAY, NJ, 08855, US

(73) Assignee: Colgate-Palmolive Company

(21) Appl. No.: 11/236,094

(22) Filed: Sep. 27, 2005

(51) Int. Cl. A61K 8/42; A61K 8/22

(52) U.S. Cl. 424/53

(57) ABSTRACT

A dual phase whitening oral care composition comprising a first phase comprising a bound peroxide in a substantially anhydrous carrier and a second phase comprising an abrasive and a tartar control system in an orally acceptably carrier. Methods of whitening a tooth surface are also provided.

### BACKGROUND OF THE INVENTION

Many individuals desire a “bright” smile and white teeth, and consider dull and stained teeth cosmetically unattractive. Unfortunately, without preventive or remedial measures, stained teeth are almost inevitable due to the absorbent nature of dental material. Everyday activities such as smoking or other oral use of tobacco products, and eating, chewing or drinking certain foods and beverages (in particular coffee, tea and red wine), cause undesirable staining of surfaces of teeth. Staining can also result from microbial activity, including that associated with dental plaque. The chromogens or color causing substances in these materials become part of the pellicle layer and can permeate the enamel layer. Even with regular brushing and flossing, years of chromogen accumulation can impart noticeable tooth discoloration.

There are a variety of compositions described in the art for preventing or treating the discoloration of teeth. In particular, to combat staining and brighten or restore the natural enamel color, a variety of products containing bleaching materials are commercially available for professional and consumer use. The materials most commonly used in teeth whitening today are peroxides. Such peroxides include hydrogen peroxide, carbamide peroxide, sodium perborate, and sodium percarbonate. When these peroxides are in appropriate contact with teeth they will usually oxidize the majority of stains, rendering the teeth whiter.

Current home whitening treatment methods include abrasive toothpastes, toothpastes that produce oxides, whitening gels for use with a dental tray and whitening strips. The effectiveness of such techniques depends on a variety of factors including the type and intensity of the stain, the type of bleaching agent, contact time of the bleaching agent on the teeth, the amount of available bleaching active in the composition, the ability of the bleaching agent to penetrate the tooth enamel, and consumer compliance.

It would be desirable to provide oral care compositions having enhanced whitening effects and superior cleaning abilities.

### BRIEF SUMMARY OF THE INVENTION

The invention provides a dual phase whitening oral care composition. The composition includes a first phase that contains a whitening agent in a substantially anhydrous and orally acceptable carrier and a second phase that contains an abrasive and an anticalculus agent in an orally acceptable carrier. The first phase and the second phase are maintained separately from each other until dispensed.

The invention further provides a method of whitening a tooth surface that includes providing the composition of the invention and contacting the first phase and the second phase of the composition so as to form an amalgam; and applying this amalgam to the tooth surface.

### DETAILED DESCRIPTION OF THE INVENTION

The present invention provides oral care compositions comprising a first phase comprising a whitening agent in a substantially anhydrous carrier; and a second phase comprising an abrasive and an anticalculus agent in an orally acceptable carrier; where the first phase and the second phase are maintained separate from each other until dispensed for application to a tooth surface. Separating the whitening agent of the first phase from the abrasive and tartar control system in the second phase allows for delivery of a highly efficacious whitening and cleaning oral care product that is shelf-stable.

The first phase comprises a whitening agent and a substantially anhydrous carrier. The total concentration of water in the first phase, including any free water and all water contained in any ingredients, is less than about 10% water by weight. This contributes to the stabilization of the whitening agent.

Preferably, the whitening agent for use in the invention includes solid whitening agents and bound whitening agents which are substantially anhydrous oxygen generating compounds. Solid whitening agents useful herein include peroxides, metal chlorites, persulfates, and combinations thereof. Exemplary peroxide phases include hydroperoxides, such as hydrogen peroxide, peroxides of alkali and alkaline earth metals, organic peroxy compounds, peroxy acids, pharmaceutically-acceptable salts thereof, and mixtures thereof. Other exemplary include peroxides of alkali and alkaline earth metals, organic peroxy compounds, peroxy acids and their salts, and as inorganic peroxy acid salts. Preferred whitening agents are sodium perborate, urea peroxide, sodium percarbonate, and mixtures thereof. Suitable metal chlorites include calcium chlorite, barium chlorite, magnesium chlorite, lithium chlorite, sodium chlorite, and potassium chlorite. The whitening agent may be preferably bound, unbound, and/or solid. For example, the whitening agent may be bound to a polymer such as PVP (poly(N-vinylpyrrolidone). Suitable PVP complexes are disclosed, for example, in U.S. Pat. Nos. 3,376,110, 3,480,557 and 5,122,370.

The first phase can optionally comprise at least one orally acceptable source of fluoride ions. Suitable sources of fluoride ions include fluoride, monofluorophosphate and fluorosilicate salts. Any such salt that is orally acceptable can be used, including without limitation alkali metal (e.g., potassium, sodium), ammonium, stannous and indium salts and the like. Water-soluble fluoride-releasing salts are typically used. Amine fluorides, including olaflur (N′-octadecyltrimethylendiamine-N,N,N′-tris(2-ethanol)-dihydrofluoride) may also be used. One or more fluoride-releasing salts are optionally present in an amount providing a total of about 100 to about 20,000 ppm, about 200 to about 5,000 ppm, or about 500 to about 2,500 ppm, fluoride ions. Where sodium fluoride is the sole fluoride-releasing salt present, it is preferably present at a level of from about 0.01% to about 5%, from about 0.05% to about 1%, or from about 0.1% to about 0.5%.

The first phase carrier is a low water content orally acceptable carrier. As used herein, an “orally acceptable carrier” refers to a material or combination of materials that are safe for use in the compositions of the present invention, commensurate with a reasonable benefit/risk ratio; with which the whitening agent, abrasive, and anticalculus agents (in the separate first and second phases and/or as mixed) may be associated while retaining significant efficacy. Preferably, the carrier does not substantially reduce the efficacy of the active materials of the present compositions.

The first phase carrier may also comprise various dentifrice ingredients to adjust the rheology and feel of the composition such as humectants, surface active agents, thickening or gelling agents, etc. It is preferred that the combination of ingredients are acidic to maintain stability of the whitening agent. Thus, in preferred embodiments, the pH of the first phase is less than about 7, more preferably from about 4 to about 6.

In various embodiments of the present invention, glycerin, propylene glycol, sorbitol, polypropylene glycol and/or polyethylene glycol (e.g., 400-600 average molecular weight) may be suitable humectants/carriers. Also advantageous are liquid mixtures of water, glycerin, and sorbitol. In various embodiments, the first phase carrier is preferably a gel comprising polyethylene glycol. Other suitable materials include PEG 400 MW, PEG 600 MW, and polymers and copolymers of PEG, of ethylene oxide, and of propylene oxide, for example, PLURAFLO® L4370 and/or L1220, each sold by BASF, Wyandotte, Mich., United States of America.

The first phase preferably comprises a surface active agent. In various embodiments, suitable surface active agents may function as a surface active agent, emulsifier, and/or foam modulator. Surface active agents generally achieve increased prophylactic action, by thoroughly dispersing the whitening agent throughout the oral cavity. Any orally acceptable surfactant, most of which are anionic, nonionic or amphoteric, can be used. Suitable anionic surfactants include without limitation water-soluble salts of C₈₋₂₀ alkyl sulfates, sulfonated monoglycerides of C₈₋₂₀ fatty acids, sarcosinates, taurates and the like. Illustrative examples of these and other classes include sodium lauryl sulfate, sodium cocoyl monoglyceride sulfonate, sodium lauryl sarcosinate, sodium lauryl isoethionate, sodium laureth carboxylate and sodium dodecyl benzenesulfonate. Suitable nonionic surfactants include without limitation poloxamers, polyoxyethylene sorbitan esters, fatty alcohol ethoxylates, alkylphenol ethoxylates, tertiary amine oxides, tertiary phosphine oxides, dialkyl sulfoxides and the like. Suitable amphoteric surfactants include without limitation derivatives of C₈₋₂₀ aliphatic secondary and tertiary amines having an anionic group such as carboxylate, sulfate, sulfonate, phosphate or phosphonate. A suitable example is cocoamidopropyl betaine.

The first phase optionally comprises a thickener. Thickeners, or gelling agents, may be selected from the group consisting of silicone fluids, carbomers, natural and synthetic gums, colloids, and mixtures thereof. In a still further embodiment a composition of the invention comprises at least one thickening agent, useful for example to impart a desired rheology, consistency, and/or mouth feel to the composition. Any orally acceptable thickening agent can be used, including without limitation carbomers, also known as carboxyvinyl polymers, carrageenans, also known as Irish moss and more particularly ι-carrageenan (iota-carrageenan), cellulosic polymers such as hydroxyethylcellulose, carboxymethylcellulose (CMC) and salts thereof, e.g., CMC sodium, natural gums such as karaya, xanthan, gum arabic and tragacanth, colloidal magnesium aluminum silicate, colloidal silica and the like. One or more thickening agents are optionally present in a total amount of about 0.1% to about 90%, for example about 1% to about 50% or about 5% to about 35% by weight of the first phase.

In various preferred embodiments, the first phase carrier comprises a mixture of polyethylene glycol, ethylene oxide propylene oxide copolymer, and silicone. The combination provides a first phase having a desirable viscosity that is temperature stable.

Any orally acceptable pH modifying agent can be included in the carrier, including carboxylic, phosphoric, and sulfonic acids, acid salts (e.g., monosodium citrate, disodium citrate, monosodium malate, etc.), alkali metal hydroxides such as sodium hydroxide, carbonates such as sodium carbonate, bicarbonates, sesquicarbonates, borates, silicates, phosphates (e.g., monosodium phosphate, trisodium phosphate, pyrophosphate salts, etc.), imidazole, and mixtures thereof. One or more pH modifying agents are optionally present in a total amount effective to maintain the composition in an orally acceptable pH range.

The second phase comprises an abrasive and an anticalculus agent in an orally acceptable carrier. Without limiting the mechanism, function or utility of present invention, it is believed that the combination of active ingredients in the second phase and the pH difference between the first and second phases assist in improved whitening efficacy and whitening agent release.

The dentally acceptable abrasive material or polishing agent may serve to either polish the tooth enamel or provide or enhance the whitening effect of the composition. Any orally acceptable abrasive can be used. Suitable abrasives include without limitation silica, for example in the form of silica gel, hydrated silica or precipitated silica, alumina, insoluble phosphates, calcium carbonate, resinous abrasives such as urea-formaldehyde condensation products and the like. Among insoluble phosphates useful as abrasives are orthophosphates, polymetaphosphates and pyrophosphates. Illustrative examples are dicalcium orthophosphate dihydrate, calcium pyrophosphate, β-calcium pyrophosphate, tricalcium phosphate, calcium polymetaphosphate and insoluble sodium polymetaphosphate. A preferred abrasive is a high cleaning silica abrasive. One or more abrasives are optionally present in an abrasive effective total amount, typically from about 0.1% to about 40% by weight of the second phase. Average particle size of an abrasive, if present, is generally about 0.1 to about 30 μm, for example about 1 to about 20 μm or about 5 to about 15 μm.

In various embodiments of the present invention, the oral composition may contain an anticalculus agent. One or more such agents can be present. Suitable anticalculus agents include any known or to be developed in the art, such as phosphates and polyphosphates (for example pyrophosphates), polyaminopropanesulfonic acid (AMPS), hexametaphosphate salts, zinc citrate trihydrate, polypeptides such as polyaspartic and polyglutamic acids, polyolefin sulfonates, polyolefin phosphates, diphosphonates such as azacycloalkane-2,2-diphosphonates (e.g., azacycloheptane-2,2-diphosphonic acid), N-methyl azacyclopentane-2,3-diphosphonic acid, ethane-1-hydroxy-1,1-diphosphonic acid (EHDP) and ethane-1-amino-1,1-diphosphonate, phosphonoalkane carboxylic acids, salts of any of these agents, for example their alkali metal and ammonium salts, and mixtures thereof.

The second phase may optionally include a peroxide activator. Peroxide activators of the present invention are preferably transition metal catalysts, alkaline compounds, or mixtures thereof. The peroxide activators accelerate the whitening effect of the composition and provide high efficacy using lower concentrations of the peroxygen compound.

If desired, a transition metal catalyst can comprise any of the stable transition elements in Groups 3 through 12 of the periodic table including cadmium, chromium, cobalt, copper, gold, hafnium, iridium, iron, lutetium, manganese, mercury, molybdenum, nickel, niobium, osmium, palladium, platinum, rhenium, rhodium, ruthenium, scandium, silver, tantalum, titanium, tungsten, vanadium, yttrium, zinc, zirconium, and combinations thereof. In particular, the transition metal catalyst can comprise iron, cobalt, nickel, copper, zinc, manganese, chromium, and combinations thereof. A preferred transition metal catalyst is manganese.

In various embodiments, the orally acceptable vehicle used to prepare the second phase of the oral care composition is a gel or paste. The humectants, surface active agents, and thickeners, as described above may be used in the second phase carrier.

Preferably, the second phase carrier also includes water. Water employed should preferably be deionized and free of organic impurities. The water is free water which is added, plus that which is introduced with other materials for example, such as that added with sorbitol. Water generally comprises from about 10% to 50%, preferably from about 20% to 40%, by weight of the second phase. The second phase carrier may also include fluoride as described above.

It is understood that the inclusion of certain ingredients may be altered depending on the pH of the respective phase and/or any potential side interactions with the active ingredients in the first and second phase as known to one of skill in the art.

As recognized by one of skill in the art, the oral compositions (both the first and/or second phases) of the present invention optionally include other materials, such as for example, anti-caries agents; desensitizing agents; viscosity modifiers; diluents; surface active agents, such as surfactants, emulsifiers, and foam modulators; pH modifying agents; abrasives, in addition to those listed above herein; humectants; mouth feel agents; sweetening agents; flavor agents; foam modulators, active agents (including pharmaceutical agents, topical or systematic agents) colorants; preservatives; and combinations thereof.

Methods are provided to whiten a tooth surface in a human or animal subject comprising maintaining a whitening oral care composition having a first phase comprising a whitening agent and a substantially anhydrous and orally acceptable carrier; and a second phase comprising an abrasive and a tartar control system in an orally acceptable carrier, where the first and second phases are separated from one another; mixing the first phase and the second phase; and contacting the mixed composition with the tooth surface. As used herein “animal subject” includes higher order non-human mammals such as canines, felines, and horses. The oral care composition is contacted with a tooth surface of the mammalian subject to thereby whiten teeth in a highly efficacious manner, without any negative interaction between the whitening agent, tartar control agent, and abrasive ingredients.

In various embodiments, it is preferred that the oral care composition is applied and contacted with the tooth surface. The dentifrice, prepared in accordance with the present invention is preferably applied regularly to a tooth surface, preferably on a daily basis, at least one time daily for multiple days, but alternately every second or third day. Preferably the oral composition is applied to the tooth surfaces from 1 to 3 times daily, at a pH of greater than about 7, preferably from about pH 8 to 10, for at least 2 weeks up to 8 weeks, from four months to three years, or more up to lifetime.

The compositions of the present invention may be packaged in any of a variety of packages, including dual compartment containers among those known in the art. Preferably, such packages contain the first phase and second phase so that the two phases are not in substantial contact until dispensing during use. In various embodiments, the first phase is stored in a first enclosure; the second phase is stored in a second enclosure; and the first phase is expelled from the first enclosure and the second phase is expelled from the second enclosure just prior to application to the teeth so that the first phase and the second phase are expelled to provide an amalgam comprising a first phase in fluid interface with a second phase. This embodiment is preferably provided to the consumer in the form of an oral care kit or package providing (a) a first chamber (the first storage enclosure for the first phase) having a first outlet in fluid communication with the first chamber for discharge of the first phase; and (b) a second chamber (the second storage enclosure for the second phase) having a second outlet in fluid communication with the second chamber for discharge of the second phase. The second outlet is proximate to the first outlet so that, the first and second phases are discharged substantially simultaneously. Such a package is also denoted herein as a dual compartment toothpaste tube. In some embodiments, approximately equal amounts of each phase are delivered into the amalgam so that the consumer has a convenient basis for ascertaining that both phases are being delivered and that rapid intermixing of the phases will occur as the amalgam is brushed against the teeth. In some embodiments, dissimilar amounts of each phase are delivered.

The invention is illustrated in the following non-limiting examples.

### EXAMPLE 1

TABLE 1

| Ingredients | 1^{st} Phase | 2^{nd} Phase |
|---|---|---|
| Ethylene oxide and Propylene oxide block | 43.557 | |
| copolymer (PLURAFLO ® L4370) | | |
| Cross-linked PVP hydrogen peroxide complex | 22.0 | |
| Sodium fluoride | 0.243 | |
| Silicone fluid 350 CST | 5.0 | |
| Ethylene oxide and Propylene oxide block | 25.0 | |
| copolymer (PLURAFLO ® L1220) | | |
| Fumed silica A200 | 2.0 | |
| Flavor | 1.2 | |
| Sodium saccharin | 1.0 | |
| Synthetic glycerin | | 12.0 |
| Sorbitol - non browning/non crystallizing | | 27.5 |
| Purified water | | 7.98 |
| Hydrated silica (SYLODENT ® 783) | | 11.0 |
| Hydrated silica (SYLODENT ® XWA 650) | | 10.0 |
| Hydrated silica (ZEODENT ® 165) | | 1.7 |
| Tetra sodium pyrophosphate | | 1.0 |
| Sodium tripolyphosphate | | 7.0 |
| Sodium carboxy methylcellulose 2000S | | 0.95 |
| Iota carrageenan | | 0.35 |
| Laponite D | | 0.75 |
| SO₃ sodium lauryl sulfate 29% | | 7.33 |
| Flavor | | 1.15 |
| Sodium saccharin | | 0.55 |
| Titanium dioxide | | 1.0 |
| Vinyl methyl ether (GANTREZ ®) | | 7.69 |
| Sodium hydroxide - 50% solution | | 2.0 |
| Manganese gluconate anhydrous | | 0.05 |
| TOTAL | 100 | 100 |

A dual phase dentifrice is prepared according to Table 1. The first phase whitening agent is a cross-linked polyvinyl pyrrolidone hydrogen peroxide complex whitening agent. The first phase carrier includes polyethylene glycol, a copolymer of ethylene oxide and propylene oxide, and a silicone fluid. The first phase carrier does not include any free water and has a proper pH to facilitate delivery of a stable PVP-hydrogen peroxide complex. Fluoride is included in the first phase to enhance oral care benefits of the dentifrice, particularly anti-caries benefits. The second phase includes the anti-calculus agents tetrasodium pyrophosphate, sodium tripolyphosphate, and vinyl methyl ether (GANTREZ®-97). The pH of the second phase is raised to a sufficiently high amount with the sodium hydroxide solution. The second phase also includes a manganese gluconate activator agent. The dual phase dentifrice provides superior cleaning and whitening benefits.

### EXAMPLE 2

A dual phase dentifrice is prepared according to Example 1. The dentifrice is stored in a dual chamber container where the first phase is separated from the second phase. The dentifrice is dispensed onto a tooth brush where the contents of the first and second phase are initially mixed together. A subject begins to brush their teeth with the dentifrice and the shear force further mixes the two phases. The oxidizing activity of the hydrogen peroxide whitening agent is released throughout mixing of the phases and provides enhanced whitening.

The examples and other embodiments described herein are exemplary and not intended to be limiting in describing the full scope of compositions and methods of this invention. Equivalent changes, modifications, and variations of specific embodiments, materials, compositions, and methods may be made within the scope of the present invention, with substantially similar results.

We claim:

1. A dual phase whitening oral care composition, comprising:
a. a first phase comprising a whitening agent in a substantially anhydrous and orally acceptable carrier; and
b. a second phase comprising an abrasive and an anticalculus agent in an orally acceptable carrier;
wherein the first phase and the second phase are maintained separately from each other until dispensed.

2. A dual phase whitening oral care composition according to claim 1, wherein the whitening agent is selected from the group consisting of bound peroxides and solid peroxides.

3. A dual phase whitening oral care composition according to claim 1, wherein the whitening agent is selected from the sodium perborate, urea peroxide, sodium percarbonate, PVP-peroxide, and sodium chlorite.

4. A dual phase whitening oral care composition according to claim 1, wherein the first phase comprises the whitening agent at a level of from about 0.1% to about 30% by weight of the first phase.

5. A dual phase whitening oral care composition according to claim 1, wherein the water content of the first phase is less than about 10% by weight of the first phase.

6. A dual phase whitening oral care composition according to claim 1, wherein the substantially anhydrous carrier comprises polymers and copolymers of PEG, ethylene oxide and propylene oxide.

7. A dual phase whitening oral care composition according to claim 1, wherein the substantially anhydrous carrier further comprises at least one of a surfactant and a thickener selected from the group consisting of silicone fluids, fumed silica, polyethylene glycol, carbomers, and gums.

8. A dual phase whitening oral care composition according to claim 1, wherein the first phase further comprises at least one agent selected from the group consisting of: peroxide, a fluoride providing agent, and mixtures thereof.

9. A dual phase whitening oral care composition according to claim 1, wherein the second phase further comprises at least one agent selected from the group consisting of a pH adjusting agent, and a fluoride providing agent.

10. A dual phase whitening oral care composition according to claim 1, wherein the abrasive of the second phase is a silica abrasive.

11. A dual phase whitening oral care composition according to claim 1, wherein the anticalculus agent of the second phase is selected from the group consisting of: inorganic phosphate salts, inorganic polyphosphate salts, polymeric polycarboxylates, sequestering agents, and mixtures thereof.

12. A dual phase whitening oral care composition according to claim 1, wherein the second phase further comprises a peroxide activator selected from the group consisting of transition metal catalysts, alkaline compounds, and combinations thereof.

13. A method of whitening a tooth surface comprising:
a. providing a whitening oral care composition having a first phase comprising a peroxide whitening agent and a substantially anhydrous and orally acceptable carrier; and a second phase comprising an abrasive and an anticalculus system in an orally acceptable carrier, wherein the first phase and the second phase are separated from one another; and
b. contacting the first phase with the second phase; and
c. applying the first phase and the second phase to the tooth surface.

14. A method of whitening a tooth surface according to claim 13, wherein the peroxide phase is a bound peroxide comprising a complex of hydrogen peroxide selected from the group consisting of poly-N-vinyl poly-2-pyrrolidone, poly-N-vinypoly-2-piperidone, poly-N-vinyl-poly-2-caprolactam, and mixtures thereof.

15. A method of whitening a tooth surface according to claim 13, wherein the peroxide is selected from the group consisting of sodium perborate, urea peroxide, sodium percarbonate, and mixtures thereof.
